(19) United States
(12) Patent Application Publication
(10) Pub. No.: US 2021/0138211 A1
(43) Pub. Date: May 13, 2021

(54) Asymmetric Articulation For Catheter Systems and Other Uses

(71) Applicant: Project Moray, Inc., Belmont, CA (US)

(72) Inventor: Keith Phillip Laby, Oakland, CA (US)

(73) Assignee: Project Moray, Inc., Belmont, CA (US)

(21) Appl. No.: 17/091,426

(22) Filed: Nov. 6, 2020

Related U.S. Application Data
(60) Provisional application No. 62/932,292, filed on Nov. 7, 2019.

(51) Int. Cl. A61M 25/10; A61M 25/00

(52) U.S. Cl. CPC A61M 25/10181; A61M 2025/1043; A61M 25/1011; A61M 25/005

(57) ABSTRACT

Image-guided interventional systems, devices, and method make use of fluid-driven articulated structures having an asymmetric cross-section. An inner sheath may be offset eccentrically within an outer sheath with an axial series of asymmetric plates disposed between them. Balloons may be mounted between adjacent plates, a series of the balloons coupled end-to-end by neck regions having smaller diameters than those of the balloons to define balloon strings. The necks can extend through channels in the plates, so that the balloons of a particular balloon string can be inflated as a unit. Ends of the balloons engage the plates where the space between the inner and outer sheaths is relatively large, and can push the adjacent plates apart with a relatively high bending force. Passages through the plates near the center of lateral bending can accommodate additional inflation fluid tubes to articulate distal segments, and a pair of articulation balloon strings may be included.

### CROSS REFERENCE TO RELATED APPLICATION DATA

The present application claims the benefit of U.S. Provisional Appln. No. 62/932,292 filed Nov. 7, 2019; the full disclosure which is incorporated herein by reference in its entirety for all purposes.

### FIELD OF THE INVENTION

In general, the present invention provides improved devices, systems, and methods for articulating an elongate flexible body such as a catheter, endoscope, or the like. The techniques provided herein will often be used to bend a therapeutic or diagnostic catheter to access and/or treat a target tissue via a constrained or tortuous access site. In exemplary embodiments, the invention provides elongate flexible bodies having articulated portions that can be driven via a fluid-drive system. Optionally, the articulated bodies may comprise heart catheters, and one of the catheters may comprise a structural heart therapy catheter, an intracoronary echocardiography (ICE) catheter, or the like.

### BACKGROUND OF THE INVENTION

Diagnosing and treating disease often involve accessing internal tissues of the human body, and open surgery is often the most straightforward approach for gaining access to internal tissues. Although open surgical techniques have been highly successful, they can impose significant trauma to collateral tissues.

To help avoid the trauma associated with open surgery, a number of minimally invasive surgical access and treatment technologies have been developed, including elongate flexible catheter structures that can be advanced along the network of blood vessel lumens extending throughout the body. While generally limiting trauma to the patient, catheter-based endoluminal therapies can be very challenging, in-part due to the difficulty in accessing (and aligning with) a target tissue using an instrument traversing tortuous vasculature. Alternative minimally invasive surgical technologies include robotic surgery, and robotic systems for manipulation of flexible catheter bodies from outside the patient have also previously been proposed. Some of those prior robotic catheter systems have met with challenges, possibly because of the difficulties in effectively integrating large and complex robotic pull-wire catheter systems into the practice of interventional cardiology as it is currently performed in clinical catheter labs. While the potential improvements to surgical accuracy make these efforts alluring, the capital equipment costs and overall burden to the healthcare system of these large, specialized systems is also a concern. Examples of prior robotic disadvantages that would be beneficial to avoid may include longer setup and overall procedure times, deleterious changes in operative modality (such as a decrease in effective tactile feedback when initially accessing or advancing tools toward an internal treatment site), and the like.

A new technology for controlling the shape of catheters has recently been proposed which may present significant advantages over pull-wires and other known catheter articulation systems. As more fully explained in U.S. Pat. No. 10,646,696, entitled “Articulation Systems, Devices, and Methods for Catheters and Other Uses,” which issued on May 12, 2020 (assigned to the assignee of the subject application and the full disclosure of which is incorporated herein by reference), an articulation balloon array can include subsets of balloons that can be inflated to selectively bend, elongate, or stiffen segments of a catheter. These articulation systems can direct pressure from a simple fluid source (such as a pre-pressurized canister) toward a subset of articulation balloons disposed along segment(s) of the catheter inside the patient so as to induce a desired change in shape. These new technologies may provide catheter control beyond what was previously available, often without having to resort to a complex robotic gantry, without having to rely on pull-wires, and even without having the expense of electric motors. Hence, these new fluid-driven catheter systems appear to provide significant advantages.

Along with the advantages of fluid-driven technologies, significant work is now underway on improved imaging for use by interventional and other doctors in guiding the movement of articulated therapy delivery systems within a patient. Ultrasound and fluoroscopy systems often acquire planar images (in some cases on different planes at angularly offset orientations), and new three-dimensional (3D) imaging technologies have been (and are still being) developed and used to show these 3D images.

Despite the advantages of the newly proposed fluid-driven robotic catheter and imaging systems, as with all successes, still further improvements and alternatives would be desirable. In general, it would be beneficial to provide further improved medical devices, systems, and methods, as well as to provide alternative devices, systems, and methods for users to direct the movements of structural heart and other image-guided interventional systems. For example, when accessing a particular tissue via the vascular pathways, one or more desirable bend orientation may be determined reliably prior to inserting an articulated catheter body into the patient. Accessing certain intravascular sites may also benefit from enhanced total bending forces an associated tight bending radii, even at the expense of bend direction capability. Hence, technologies which facilitate precise image-guided movements of interventional tools—including those mounted on relatively stiff shafts—would be particularly beneficial.

### BRIEF SUMMARY OF THE INVENTION

The present invention generally provides improved devices, systems, and methods for using fluid to articulate a catheter or other elongate flexible body. The structures described herein will often include an asymmetric cross-section, with an inner sheath being offset eccentrically within a surrounding outer sheath. An axial series of asymmetric plates may be disposed between (and maintain eccentric alignment of) the offset inner and outer sheaths, and balloons may be mounted between adjacent plates. The balloons may be included in a balloon string with a series of balloons coupled end-to-end by neck regions having smaller diameters than those of the balloons. The necks can extend through channels in the plates, so that the balloons of a particular balloon string can be inflated as a unit. Ends of the balloons can engage the plates where the space between the inner and outer sheaths is relatively large, and can push the adjacent plates apart with a relatively high bending force by taking advantage of the large balloon end/plate engagement area and lateral offset between the associated axial load and the center of the inner sheath. Passages through the plates near the center of lateral bending can accommodate additional inflation fluid tubes to articulate distal segments, and a pair of articulation balloon strings may be included in each of one or more asymmetrical segments of a catheter.

In a first aspect, the invention provides a fluid-articulated structure comprising an inner sheath having a proximal end and a distal end with an inner lumen extending therebetween. The inner sheath may have an inner sheath axis, and an axial series of asymmetric plates may extend radially outwardly from the inner sheath. The asymmetric plates can have opposed axial surfaces, the axial surfaces having eccentric apertures with the inner lumen of the inner sheath extending therethrough so that more than half of the area of the axial surfaces are on a first lateral side of the asymmetric plates relative to the inner sheath. The asymmetric plates can have channels extending between the major surfaces on the first lateral side. A balloon string may also be included, which may have an axial series of balloons separated by necks. The necks can extend through the channels of the asymmetric plates and the balloons can have axially oriented ends engaging the major surfaces on the first lateral sides of the asymmetric plates so that when the balloons are inflated the balloons urge the first sides of the plates apart and bend the inner sheath axis.

A variety of addition independent features may also be included, alone or in combination, to enhance utility of the structures and methods described herein. Optionally, an outer sheath with an outer sheath lumen can be included, the outer sheath also having an outer axis extending along the centerline of the outer lumen. The outer lumen may receive the asymmetric plates and inner sheath therein so that the inner lumen of the inner sheath is offset eccentrically relative to the outer lumen of the outer sheath. The balloons will often engage an inner surface of the outer sheath. The outer sheath will often comprise a polymer and a reinforcing member extending circumferentially around the outer lumen. The reinforcing member can be configured to inhibit radial dilation of the outer sheath when the balloon is inflated and the polymer of the outer sheath stretches to accommodate axial elongation along the first side of the outer sheath between the plates. Optionally, the inner sheath comprises a polymer and a reinforcing member extending circumferentially around the inner lumen, with the reinforcing member of the inner sheath configured to inhibit radial compression of the inner lumen when the balloon is inflated and the polymer of the inner sheath flexes to accommodate axial bending between the plates. When both are included, inflation of the balloons can induce loading of the reinforcing member of the outer sheath in tension and loading of the reinforcing member of the inner sheath in compression and bending, the reinforcing member of the inner sheath optionally having a first stiffness and the reinforcing member of the outer sheath having a second stiffness, the second stiffness being less than the first stiffness. For example, the reinforcing member of the inner sheath may comprise a coil or braid with a first metal wire having a first diameter, and the reinforcing member of the outer sheath may comprise a coil or braid with a second metal wire having a second diameter smaller than the first diameter.

Other independent features include that the asymmetric plates may be torsionally affixed to the inner sheath or the outer sheath or both, optionally by adhesive bonding, ultrasound welding, or thermal bonding. The channels of the asymmetric plates can extend laterally to the aperture or a peripheral edge to facilitate insertion of the balloon strings, such as by passing one or more of the relatively large diameter balloons of the string through the aperture till a desired neck is aligned with a channel in a particular plate, and then moving the neck laterally into the channel.

Optionally, first and second balloon strings may extends axially through associated channels of the asymmetric plates, each balloon string having a plurality of balloons being disposed between adjacent asymmetric plates and, when inflated, urging the first sides of the plates apart to induce bending. The first and second balloon strings can be in fluid communication so as to be inflated together at a common inflation pressure, typically providing a single bending degree of freedom for that particular segment. Alternatively, the two balloon strings may be independently to different pressures so as to allow some transverse lateral articulation (along with bending in a first lateral bending orientation away from the large first side of the asymmetric plate), such transverse articulation optionally having a smaller range of movement than the first bending orientation.

Advantageously, the asymmetric plates, associated balloons, and the like can be included in a first (often a proximal) segment of a catheter having multiple independently articulatable segments. Optionally, the asymmetric plates can have passages extending between the axial surfaces, and the proximal segment can be configured to bend laterally in a first bending orientation oriented away from the first sides of the axial plates. An inflation fluid supply tube may extend axially through the passages, and a distal segment can have a balloon array in fluid communication with the inflation fluid supply tube so as to bend laterally in a second lateral bending orientation transverse to the first lateral bend orientation. Optionally, the balloon array of the second segment can be configured to bend laterally in a plurality of orientations, the inflation fluid supply tube having a plurality of lumens. Each asymmetric plate may have a plurality of passages and a plurality of inflation fluid supply tubes can extend axially therethrough so as to allow bending of the overall structure at a plurality of axially offset segments.

In another aspect, the invention provides a fluid-articulated structure comprising an outer sheath having a proximal end and a distal end with a lumen extending therebetween. The lumen of the outer sheath can have a central axis. An inner sheath can be disposed within the lumen of the outer sheath, the inner sheath having an inner lumen and an offset axis which is disposed in the center of the inner lumen, but which is offset eccentrically relative to the central axis of the outer sheath. An axial series of asymmetric plates can be disposed between the inner sheath and the outer sheath, the asymmetric plates having opposed axial surfaces. The axial surfaces can have eccentric apertures receiving the inner sheath therethrough so the axial surfaces are primarily on a first lateral side of the plates relative to the inner sheath. The asymmetric plates can have channels extending between the major surfaces on the first lateral side. A balloon string may extend axially, and may include an axial series of balloons separated by necks. The necks may extend through the channels of the asymmetric plates and the balloons may have axially oriented ends engaging the major surfaces on the first lateral sides of the asymmetric plates so that when the balloons are inflated the balloons urge the plates apart and bend the central axis of the outer sheath.

In a method aspect, the invention provides a method for bending a fluid-articulated structure. The method comprises inflating a balloon string including an axial series of balloons separated by necks. The necks can extend through channels of a plurality of asymmetric plates, and the asymmetric plates can have eccentric apertures with an inner sheath extending therethrough. The inflating can be performed so as to urge axially oriented ends of the balloons against major surfaces of the asymmetric plates on first lateral sides of the asymmetric plates relative to the sheath. The inflation of the balloons can urge the first sides of the plates apart and can bend a central axis of the inner sheath.

### BRIEF DESCRIPTION OF THE DRAWINGS

FIG. 1 illustrates an interventional cardiologist performing a structural heart procedure with a robotic catheter system having a fluidic catheter driver slidably supported by a stand.

FIG. 2 is a simplified schematic illustration of components of a helical balloon assembly, showing how an extruded multi-lumen shaft can provide fluid to laterally aligned subsets of balloons within an articulation balloon array of a catheter.

FIGS. 3A-3C schematically illustrate helical balloon assemblies supported by flat springs and embedded in an elastomeric polymer matrix, and also show how selective inflation of subsets of the balloons can elongate and laterally articulate the assemblies.

FIG. 4 is a perspective view of a robotic catheter system in which a catheter is removably mounted on a driver assembly, and in which the driver assembly includes a driver encased in a sterile housing and supported by a stand.

FIG. 5 schematically illustrates a robotic catheter system and transmission of signals between the components thereof so that input from a user induces a desired articulation.

FIGS. 6A-6C are a side, bottom, and end view, respectively, of an asymmetric balloon articulation assembly, with axial portions of the assembly omitted to help show the underlying structures, with the assembly shown in a straight configuration.

FIGS. 6D and 6E are alternative perspective views of the assembly of FIGS. 6A-6C, with the assembly shown in an articulated (bent) configuration.

FIGS. 6F, 6G and 6H are a front, side, and cross-sectional view of the assembly of FIGS. 6D and 6E.

FIGS. 7A and 7B are a top view and a perspective view, respectively, of an asymmetric plate used in the assembly of FIGS. 6A-6C.

FIG. 8 is a perspective view of a sub-assembly used in the assembly of FIGS. 6A-6C, showing how the balloon strings are assembled into and interact with the asymmetric plates.

### DETAILED DESCRIPTION OF THE INVENTION

The improved devices, systems, and methods for controlling, image guidance of, inputting commands into, and simulating movement of powered and robotic devices will find a wide variety of uses. The elongate tool-supporting structures described herein will often be flexible, typically comprising catheters suitable for insertion in a patient body. Exemplary systems will be configured for insertion into the vascular system, the systems typically including a cardiac catheter and supporting a structural heart tool for repairing or replacing a valve of the heart, occluding an ostium or passage, or the like. Other cardiac catheter systems will be configured for diagnosis and/or treatment of congenital defects of the heart, or may comprise electrophysiology catheters configured for diagnosing or inhibiting arrhythmias (optionally by ablating a pattern of tissue bordering or near a heart chamber).

Alternative applications may include use in steerable supports of image acquisition devices such as for trans-esophageal echocardiography (TEE), intra-coronary echocardiography (ICE), and other ultrasound techniques, endoscopy, and the like. The structures described herein will often find applications for diagnosing or treating the disease states of or adjacent to the cardiovascular system, the alimentary tract, the airways, the urogenital system, and/or other lumen systems of a patient body. Other medical tools making use of the articulation systems described herein may be configured for endoscopic procedures, or even for open surgical procedures, such as for supporting, moving and aligning image capture devices, other sensor systems, or energy delivery tools, for tissue retraction or support, for therapeutic tissue remodeling tools, or the like. Alternative elongate flexible bodies that include the articulation technologies described herein may find applications in industrial applications (such as for electronic device assembly or test equipment, for orienting and positioning image acquisition devices, or the like). Still further elongate articulatable devices embodying the techniques described herein may be configured for use in consumer products, for retail applications, for entertainment, or the like, and wherever it is desirable to provide simple articulated assemblies with one or more (preferably multiple) degrees of freedom without having to resort to complex rigid linkages.

Embodiments provided herein may use balloon-like structures to effect articulation of the elongate catheter or other body. The term “articulation balloon” may be used to refer to a component which expands on inflation with a fluid and is arranged so that on expansion the primary effect is to cause articulation of the elongate body. Note that this use of such a structure is contrasted with a conventional interventional balloon whose primary effect on expansion is to cause substantial radially outward expansion from the outer profile of the overall device, for example to dilate or occlude or anchor in a vessel in which the device is located. Independently, articulated medial structures described herein will often have an articulated distal portion, and an unarticulated proximal portion, which may significantly simplify initial advancement of the structure into a patient using standard catheterization techniques.

The robotic systems described herein will often include an input device, a driver, and an articulated catheter or other robotic manipulator supporting a diagnostic or therapeutic tool. The user will typically input commands into the input device, which will generate and transmit corresponding input command signals. The driver will generally provide both power for and articulation movement control over the tool. Hence, somewhat analogous to a motor driver, the driver structures described herein will receive the input command signals from the input device and will output drive signals to the tool-supporting articulated structure so as to effect robotic movement of an articulated feature of the tool (such as movement of one or more laterally deflectable segments of a catheter in multiple degrees of freedom). The drive signals may comprise fluidic commands, such as pressurized pneumatic or hydraulic flows transmitted from the driver to the tool-supporting catheter along a plurality of fluid channels. Optionally, the drive signals may comprise electromagnetic, optical, or other signals, preferably (although not necessarily) in combination with fluidic drive signals. Unlike many robotic systems, the robotic tool supporting structure will often (though not always) have a passively flexible portion between the articulated feature (typically disposed along a distal portion of a catheter or other tool manipulator) and the driver (typically coupled to a proximal end of the catheter or tool manipulator). The system will be driven while sufficient environmental forces are imposed against the tool or catheter to impose one or more bend along this passive proximal portion, the system often being configured for use with the bend(s) resiliently deflecting an axis of the catheter or other tool manipulator by 10 degrees or more, more than 20 degrees, or even more than 45 degrees.

The catheter bodies (and many of the other elongate flexible bodies that benefit from the inventions described herein) will often be described herein as having or defining an axis, such that the axis extends along the elongate length of the body. As the bodies are flexible, the local orientation of this axis may vary along the length of the body, and while the axis will often be a central axis defined at or near a center of a cross-section of the body, eccentric axes near an outer surface of the body might also be used. It should be understood, for example, that an elongate structure that extends “along an axis” may have its longest dimension extending in an orientation that has a significant axial component, but the length of that structure need not be precisely parallel to the axis. Similarly, an elongate structure that extends “primarily along the axis” and the like will generally have a length that extends along an orientation that has a greater axial component than components in other orientations orthogonal to the axis. Other orientations may be defined relative to the axis of the body, including orientations that are transvers to the axis (which will encompass orientation that generally extend across the axis, but need not be orthogonal to the axis), orientations that are lateral to the axis (which will encompass orientations that have a significant radial component relative to the axis), orientations that are circumferential relative to the axis (which will encompass orientations that extend around the axis), and the like. The orientations of surfaces may be described herein by reference to the normal of the surface extending away from the structure underlying the surface. As an example, in a simple, solid cylindrical body that has an axis that extends from a proximal end of the body to the distal end of the body, the distal-most end of the body may be described as being distally oriented, the proximal end may be described as being proximally oriented, and the curved outer surface of the cylinder between the proximal and distal ends may be described as being radially oriented. As another example, an elongate helical structure extending axially around the above cylindrical body, with the helical structure comprising a wire with a square cross section wrapped around the cylinder at a 20 degree angle, might be described herein as having two opposed axial surfaces (with one being primarily proximally oriented, one being primarily distally oriented). The outermost surface of that wire might be described as being oriented exactly radially outwardly, while the opposed inner surface of the wire might be described as being oriented radially inwardly, and so forth.

Referring first to FIG. 1, a system user U, such as an interventional cardiologist, uses a robotic catheter system 10 to perform a procedure in a heart H of a patient P. System 10 generally includes an articulated catheter 12, a driver assembly 14, and an input device 16. User U controls the position and orientation of a therapeutic or diagnostic tool mounted on a distal end of catheter 12 by entering movement commands into input 16, and optionally by sliding the catheter relative to a stand of the driver assembly, while viewing a distal end of the catheter and the surrounding tissue in a display D. As will be described below, user U may alternatively manually rotate the catheter body about its axis in some embodiments.

During use, catheter 12 extends distally from driver system 14 through a vascular access site S, optionally (though not necessarily) using an introducer sheath. A sterile field 18 encompasses access site S, catheter 12, and some or all of an outer surface of driver assembly 14. Driver assembly 14 will generally include components that power automated movement of the distal end of catheter 12 within patient P, with at least a portion of the power often being transmitted along the catheter body as a hydraulic or pneumatic fluid flow. To facilitate movement of a catheter-mounted therapeutic tool per the commands of user U, system 10 will typically include data processing circuitry, often including a processor within the driver assembly. Regarding that processor and the other data processing components of system 10, a wide variety of data processing architectures may be employed. The processor, associated pressure and/or position sensors of the driver assembly, and data input device 16, optionally together with any additional general purpose or proprietary computing device (such as a desktop PC, notebook PC, tablet, server, remote computing or interface device, or the like) will generally include a combination of data processing hardware and software, with the hardware including an input, an output (such as a sound generator, indicator lights, printer, and/or an image display), and one or more processor board(s). These components are included in a processor system capable of performing the transformations, kinematic analysis, and matrix processing functionality associated with generating the valve commands, along with the appropriate connectors, conductors, wireless telemetry, and the like. The processing capabilities may be centralized in a single processor board, or may be distributed among various components so that smaller volumes of higher-level data can be transmitted. The processor(s) will often include one or more memory or other form of volatile or non-volatile storage media, and the functionality used to perform the methods described herein will often include software or firmware embodied therein. The software will typically comprise machine-readable programming code or instructions embodied in non-volatile media and may be arranged in a wide variety of alternative code architectures, varying from a single monolithic code running on a single processor to a large number of specialized subroutines, classes, or objects being run in parallel on a number of separate processor sub-units.

Referring still to FIG. 1, along with display D, a simulation display SD may present an image of an articulated portion of a simulated or virtual catheter S12 with a receptacle for supporting a simulated therapeutic or diagnostic tool. The simulated image shown on the simulation display SD may optionally include a tissue image based on pre-treatment imaging, intra-treatment imaging, and/or a simplified virtual tissue model, or the virtual catheter may be displayed without tissue. Simulation display SD may have or be included in an associated computer 15, and the computer will preferably be couplable with a network and/or a cloud 17 so as to facilitate updating of the system, uploading of treatment and/or simulation data for use in data analytics, and the like. Computer 15 may have a wireless, wired, or optical connection with input device 16, a processor of driver assembly 14, display D, and/or cloud 17, with suitable wireless connections comprising a Bluetooth™ connection, a WiFi connection, or the like. Preferably, an orientation and other characteristics of simulated catheter S12 may be controlled by the user U via input device 16 or another input device of computer 15, and/or by software of the computer so as to present the simulated catheter to the user with an orientation corresponding to the orientation of the actual catheter as sensed by a remote imaging system (typically a fluoroscopic imaging system, an ultra-sound imaging system, a magnetic resonance imaging system (MRI), or the like) incorporating display D and an image capture device 19. Optionally, computer 15 may superimpose an image of simulated catheter S12 on the tissue image shown by display D (instead of or in addition to displaying the simulated catheter on simulation display SD), preferably with the image of the simulated catheter being registered with the image of the tissue and/or with an image of the actual catheter structure in the surgical site. Still other alternatives may be provided, including presenting a simulation window showing simulated catheter SD on display D, including the simulation data processing capabilities of computer 15 in a processor of driver assembly 14 and/or input device 16 (with the input device optionally taking the form of a tablet that can be supported by or near driver assembly 14, incorporating the input device, computer, and one or both of displays D, SD into a workstation near the patient, shielded from the imaging system, and/or remote from the patient, or the like.

Referring now to FIG. 2, the components of, and fabrication method for production of, an exemplary balloon array assembly (sometimes referred to herein as a balloon string 32) can be understood. A multi-lumen shaft 34 will typically have between 3 and 18 lumens. The shaft can be formed by extrusion with a polymer such as a nylon, a polyurethane, a thermoplastic such as a Pebax™ thermoplastic or a polyether ether ketone (PEEK) thermoplastic, a polyethylene terephthalate (PET) polymer, a polytetrafluoroethylene (PTFE) polymer, or the like. A series of ports 36 are formed between the outer surface of shaft 36 and the lumens, and a continuous balloon tube 38 is slid over the shaft and ports, with the ports being disposed in large profile regions of the tube and the tube being sealed over the shaft along the small profile regions of the tube between ports to form a series of balloons. The balloon tube may be formed using a compliant, non-compliant, or semi-compliant balloon material such as a latex, a silicone, a nylon elastomer, a polyurethane, a nylon, a thermoplastic such as a Pebax™ thermoplastic or a polyether ether ketone (PEEK) thermoplastic, a polyethylene terephthalate (PET) polymer, a polytetrafluoroethylene (PTFE) polymer, or the like, with the large-profile regions preferably being blown sequentially or simultaneously to provide desired hoop strength. The ports can be formed by laser drilling or mechanical skiving of the multi-lumen shaft with a mandrel in the lumens. Each lumen of the shaft may be associated with between 3 and 50 balloons, typically from about 5 to about 30 balloons. The shaft balloon assembly 40 can be coiled to a helical balloon array of balloon string 32, with one subset of balloons 42a being aligned along one side of the helical axis 44, another subset of balloons 44b (typically offset from the first set by 120 degrees) aligned along another side, and a third set (shown schematically as deflated) along a third side. Alternative embodiments may have four subsets of balloons arranged in quadrature about axis 44, with 90 degrees between adjacent sets of balloons.

Referring now to FIGS. 3A, 3B, and 3C, an articulated segment assembly 50 has a plurality of helical balloon strings 32, 32′ arranged in a double helix configuration. A pair of flat springs 52 are interleaved between the balloon strings and can help axially compress the assembly and urge deflation of the balloons. As can be understood by a comparison of FIGS. 3A and 3B, inflation of subsets of the balloons surrounding the axis of segment 50 can induce axial elongation of the segment. As can be understood with reference to FIGS. 3A and 3C, selective inflation of a balloon subset 42a offset from the segment axis 44 along a common lateral bending orientation X induces lateral bending of the axis 44 away from the inflated balloons. Variable inflation of three or four subsets of balloons (via three or four channels of a single multi-lumen shaft, for example) can provide control over the articulation of segment 50 in three degrees of freedom, i.e., lateral bending in the +/−X orientation and the +/−Y orientation, and elongation in the +Z orientation. As noted above, each multilumen shaft of the balloon strings 32, 32′ may have more than three channels (with the exemplary shafts having 6 or 7 lumens), so that the total balloon array may include a series of independently articulatable segments (each having 3 or 4 dedicated lumens of one of the multi-lumen shafts, for example). Optionally, from 2 to 4 modular, axially sequential segments may each have an associated tri-lumen shaft, with the tri-lumen shaft extending axially in a loose helical coil through the lumen of any proximal segments to accommodate bending and elongation. The segments may each include a single helical balloon string/multilumen shaft assembly (rather than having a dual-helix configuration). Multi-lumen shafts for driving of distal segments may alternatively wind proximally around an outer surface of a proximal segment, or may be wound parallel and next to the multi-lumen shaft/balloon tube assemblies of the balloon array of the proximal segment(s).

Referring still to FIGS. 3A, 3B, and 3C, articulated segment 50 optionally includes a polymer matrix 54, with some or all of the outer surface of balloon strings 32, 32′ and flat springs 52 that are included in the segment being covered by the matrix. Matrix 54 may comprise, for example, a relatively soft elastomer to accommodate inflation of the balloons and associated articulation of the segment, with the matrix optionally helping to urge the balloons toward an at least nominally deflated state, and to urge the segment toward a straight, minimal length configuration. Alternatively (or in addition to a relatively soft matrix), a thin layer of relatively high-strength elastomer can be applied to the assembly (prior to, after, or instead of the soft matrix), optionally while the balloons are in an at least partially inflated state. Advantageously, matrix 54 can help maintain overall alignment of the balloon array and springs within the segment despite segment articulation and bending of the segment by environmental forces. Regardless of whether or not a matrix is included, an inner sheath may extend along the inner surface of the helical assembly, and an outer sheath may extend along an outer surface of the assembly, with the inner and/or outer sheaths optionally comprising a polymer reinforced with wire or a high-strength fiber in a coiled, braid, or other circumferential configuration to provide hoop strength while accommodating lateral bending (and preferably axial elongation as well). The inner and outer sheaths may be sealed together distal of the balloon assembly, forming an annular chamber with the balloon array disposed therein. A passage may extend proximally from the annular space around the balloons to the proximal end of the catheter to safely vent any escaping inflation media, or a vacuum may be drawn in the annular space and monitored electronically with a pressure sensor to inhibit inflation flow if the vacuum deteriorates.

Referring now to FIG. 4, a proximal housing 62 of catheter 12 and the primary components of driver assembly 14 can be seen in more detail. Catheter 12 generally includes a catheter body 64 that extends from proximal housing 62 to an articulated distal portion 66 (see FIG. 1) along an axis 67, with the articulated distal portion preferably comprising a balloon array and the associated structures described above. Proximal housing 62 also contains first and second rotating latch receptacles 68a, 68b which allow a quick-disconnect removal and replacement of the catheter. The components of driver assembly 14 visible in FIG. 4 include a sterile housing 70 and a stand 72, with the stand supporting the sterile housing so that the sterile housing (and components of the driver assembly therein, including the driver) and catheter 12 can move axially along axis 67. Sterile housing 70 generally includes a lower housing 74 and a sterile junction having a sterile barrier 76. Sterile junction 76 releasably latches to lower housing 74 and includes a sterile barrier body that extends between catheter 12 and the driver contained within the sterile housing. Along with components that allow articulation fluid flow to pass through the sterile fluidic junction, the sterile barrier may also include one or more electrical connectors or contacts to facilitate data and/or electrical power transmission between the catheter and driver, such as for articulation feedback sensing, manual articulations sensing, or the like. The sterile housing 70 will often comprise a polymer such as an ABS plastic, a polycarbonate, acetal, polystyrene, polypropylene, or the like, and may be injection molded, blow molded, thermoformed, 3-D printed, or formed using still other techniques. Polymer sterile housings may be disposable after use on a single patient, may be sterilizable for use with a limited number of patients, or may be sterilizable indefinitely; alternative sterile housings may comprise metal for long-term repeated sterile processing. Stand 72 will often comprise a metal, such as a stainless steel, aluminum, or the like for repeated sterilizing and use.

Referring now to FIG. 5, components of a simulation system 101 that can be used for simulation, training, pre-treatment planning, and or treatment of a patent are schematically illustrated. Some or all of the components of system 101 may be used in addition to or instead of the clinical components of the system shown in FIG. 1. System 101 may optionally include an alternative catheter 112 and an alternative driver assembly 114, with the alternative catheter comprising a real and/or virtual catheter and the driver assembly comprising a real and/or virtual driver 114.

Alternative catheter 112 can be replaceably coupled with alternative driver assembly 114. When simulation system 101 is used for driving an actual catheter, the coupling may be performed using a quick-release engagement between an interface 113 on a proximal housing of the catheter and a catheter receptacle 103 of the driver assembly. An elongate body 105 of catheter 112 has a proximal/distal axis as described above and a distal receptacle 107 that is configured to support a therapeutic or diagnostic tool 109 such as a structural heart tool for repairing or replacing a valve of a heart. The tool receptacle may comprise an axial lumen for receiving the tool within or through the catheter body, a surface of the body to which the tool is permanently affixed, or the like. Alternative drive assembly 114 may be wireless coupled to a simulation computer 115 and/or a simulation input device 116, or cables may be used for transmission of data.

When alternative catheter 112 and alternative drive system 114 comprise virtual structures, they may be embodied as modules of software, firmware, and/or hardware. The modules may optionally be configured for performing articulation calculations modeling performance of some or all of the actual clinical components as described below, and/or may be embodied as a series of look-up tables to allow computer 115 to generate a display effectively representing the performance. The modules will optionally be embodied at least in-part in a non-volatile memory of a simulation-supporting alternative drive assembly 121a, but some or all of the simulation modules will preferably be embodied as software in non-volatile memories 121b, 121c of simulation computer 115 and/or simulation input device 116, respectively. Coupling of alternative virtual catheters and tools can be permed using menu options or the like. In some embodiments, selection of a virtual catheter may be facilitated by a signal generated in response to mounting of an actual catheter to an actual driver.

Simulation computer 115 preferably comprises an off-the-shelf notebook or desktop computer that can be coupled to cloud 17, optionally via an intranet, the internet, an ethernet, or the like, typically using a wireless router or a cable coupling the simulation computer to a server. Cloud 17 will preferably provide data communication between simulation computer 115 and a remote server, with the remote server also being in communication with a processor of other simulation computers 115 and/or one or more clinical drive assemblies 14. Simulation computer 115 may also comprise code with a virtual 3D workspace, the workspace optionally being generated using a proprietary or commercially available 3D development engine that can also be used for developing games and the like, such as Unity™ as commercialized by Unity Technologies. Suitable off-the-shelf computers may include any of a variety of operating systems (such as Windows from Microsoft, OS from Apple, Linex, or the like), along with a variety of additional proprietary and commercially available apps and programs.

Simulation input device 116 may comprise an off-the-shelf input device having a sensor system for measuring input commands in at least two degrees of freedom, preferably in 3 or more degrees of freedom, and in some cases 5, 6, or more degrees of freedom. Suitable off-the-shelf input devices include a mouse (optionally with a scroll wheel or the like to facilitate input in a 3^{rd} degree of freedom), a tablet or phone having an X-Y touch screen (optionally with AR capabilities such as being compliant with ARCor from Google, ARKit from Apple, or the like to facilitate input of translation and/or rotation, along with multi-finger gestures such as pinching, rotation, and the like), a gamepad, a 3D mouse, a 3D stylus, or the like. Proprietary code may be loaded on the simulation input device (particularly when a phone, tablet, or other device having a touchscreen is used), with such input device code presenting menu options for inputting additional commands and changing modes of operation of the simulation or clinical system. A simulation input/output system 111 may be defined by the simulation input device 116 and the simulation display SD.

Referring now to FIGS. 6A-6C, an exemplary asymmetric fluid-articulated segment structure 150 that may be included in a catheter system with helical segments similar to those described above, or alone, or with an axial series of other asymmetric segments, or with still further alternative articulated segments (such as pull-wires or the like). Fluid-articulated structure 150 includes an inner sheath 152 having a proximal end 154 and a distal end 156 with an inner lumen 158 extending therebetween, the inner sheath having an inner sheath axis 160. Inner sheath axis 160 may be a central axis extending along a center of inner lumen 158. An outer sheath 165 may similarly extend between proximal end 154 and a distal end 156 with an outer sheath lumen 168 extending therebetween. The lumen 168 of the outer sheath 166 may have an outer sheath central axis 170. As the inner and outer sheaths 152, 166 are eccentric, inner axis 160 may be offset relative to outer axis 170. An axial series 162 of asymmetric plates 164 typically extend between inner sheath 152 and outer sheath 166.

As seen most clearly in FIGS. 7A and 7B, asymmetric plates 164 have opposed axial surfaces 176a, 176b, the axial surfaces having eccentric apertures 178 in which the inner sheath is disposed. As apertures 178 are eccentric, axial surfaces 176a, 176b are primarily on a first lateral side 180 of the asymmetric plates 166 relative to the axis 160 of the inner sheath 152. The asymmetric plates 166 also have channels 182 extending between the major surfaces 176a, 176b on the first lateral side 180, with the channels preferably being open to an outer perimeter edge 184 or an inner aperture edge 186 to facilitate assembly. Preferably, asymmetric plates 166 also have one or more passages 188 extending between axial surfaces 176a, 176b, with the passages again being open to an inner or outer edge of the plate. Passages 188 receive fluid supply tubes 192 which provide inflation fluid for balloon articulation of more distal segments (or other structures use to articulate or deploy the catheter distal of fluid-articulated structure 150) and are preferably positioned near or along a lateral bend line extending across plates 166 between first side 180 and a second side 190 of the plates so that a total axial length of the path defined by the passages of the asymmetric plates along the segment remains roughly constant when the segment articulates.

Referring now to FIGS. 6B, 6F, 7A, and 8, first and second balloon strings 194a, 194b extend axially through the channels 188 of plates 166. Each balloon string includes an axial series of balloons 196 separated by necks 198, the necks extending through the channels 182 of the asymmetric plates 166 and the balloons having axially oriented ends 200 engaging the major or axial surfaces 176a, 176b on the first lateral sides 180 of the asymmetric plates 166. The length of the balloons between the balloon ends may be, when the balloons are inflated to an actuation pressure, longer than the nominal distance between adjacent axial surfaces of the asymmetric plates. As a result, when the balloon strings (and hence the balloons) are inflated the balloons urge the first sides 180 of the plates 166 apart and bend segment.

Balloons 196 may be formed as cylinders, but may be constrained by the inner and/or outer sheaths to have a non-round axial cross-section. The articulation may be a factor of the pressure within the balloons, the engagement area between the balloons and the asymmetric plates, and the offset distance or moment arm between the center of balloon/plate engagement and the center of bending of the segment. While inner and outer sheaths 152, 165 both preferably constrain the balloons, the nature of that constraint is somewhat different: the balloons engage the outer sheath and push radially outwardly, loading the outer sheath in tension. Hence, when the outer sheath comprises a polymer and a reinforcing member extending circumferentially around the outer lumen, the reinforcing member can be configured to inhibit radial dilation of the outer sheath when the balloon is inflated and the polymer of the outer sheath can stretch to accommodate axial elongation along the first side of the outer sheath between the plates. In contrast, when the inner sheath comprises a polymer and a reinforcing member extending circumferentially around the inner lumen, the reinforcing member of the inner sheath should be configured to inhibit radial compression of the inner lumen when the balloon is inflated and the polymer of the inner sheath flexes to accommodate axial bending between the plates. As a result, the reinforcing member of the inner sheath may benefit from a stiffness that is greater than the stiffness of the reinforcing member of the outer sheath. For example, when the reinforcing member of the inner sheath comprises a coil or braid with a first metal wire having a first diameter, the reinforcing member of the outer sheath may comprise a coil or braid with a second metal wire having a second diameter smaller than the first diameter.

Bending of asymmetric fluid-driven structure 150 is seen in FIGS. 6D-6G. Along with a significant bending moment, inflation of the balloons may induce some torsion between asymmetric plates 166 around the axis of the inner or outer sheath 152, 165. An interference fit between the plates and the inner or outer sheath may be sufficient to inhibit torsional displacement of the assembly. Alternatively, the asymmetric plates may be torsionally affixed to the inner sheath or the outer sheath or both by bonds 208, with the bonds optionally being formed by adhesive bonding, ultrasound welding, thermal bonding, or the like. More generally, bending of fluid-articulated structure 150 may be performed by inflating a balloon string extending through channels of a plurality of asymmetric plates 166, the asymmetric plates having eccentric apertures with an inner sheath extending therethrough. The inflating can be performed so as to urge axially oriented ends of the balloons against major surfaces of the asymmetric plates on first lateral sides 180 of the asymmetric plates relative to the inner sheath, and the balloons can urge the first sides of the plates apart so as to bend a central axis of the inner sheath.

While the exemplary embodiments have been described in some detail for clarity of understanding and by way of example, a variety of modifications, changes, and adaptations of the structures and methods described herein will be obvious to those of skill in the art. Hence, the scope of the present invention is limited solely by the claims attached hereto.

What is claimed is:

1. A fluid-articulated structure comprising:
an inner sheath having a proximal end and a distal end with an inner lumen extending therebetween, the inner sheath having an inner sheath axis;
an axial series of asymmetric plates extending radially outwardly from the inner sheath, the asymmetric plates having opposed axial surfaces, the axial surfaces having eccentric apertures with the inner lumen of the inner sheath extending therethrough so the axial surfaces are primarily on a first lateral side of the asymmetric plates relative to the inner sheath, the asymmetric plates having channels extending between the major surfaces on the first lateral side; and
a balloon string including an axial series of balloons separated by necks, the necks extending through the channels of the asymmetric plates and the balloons having axially oriented ends engaging the major surfaces on the first lateral sides of the asymmetric plates so that when the balloons are inflated the balloons urge the first sides of the plates apart and bend the inner sheath axis.

2. The fluid-articulated structure of claim 1, further comprising an outer sheath with an outer sheath lumen having an outer axis, the outer lumen receiving the asymmetric plates and inner sheath therein so that the inner lumen of the inner sheath is offset eccentrically relative to the outer lumen of the outer sheath.

3. The fluid-articulated structure of claim 2, wherein the balloons engage the outer sheath, wherein the outer sheath comprises a polymer and a reinforcing member extending circumferentially around the outer lumen, the reinforcing member configured to inhibit radial dilation of the outer sheath when the balloon is inflated and the polymer of the outer sheath stretches to accommodate axial elongation along the first side of the outer sheath between the plates.

4. The fluid-articulated structure of claim 2, wherein the inner sheath comprises a polymer and a reinforcing member extending circumferentially around the inner lumen, the reinforcing member of the inner sheath configured to inhibit radial compression of the inner lumen when the balloon is inflated and the polymer of the inner sheath flexes to accommodate axial bending between the plates.

5. The fluid-articulated structure of claim 4, wherein inflation of the balloons induces loading of the reinforcing member of the outer sheath in tension and loading of the reinforcing member of the inner sheath in compression and bending, the reinforcing member of the inner sheath having a first stiffness and the reinforcing member of the outer sheath having a second stiffness, the second stiffness being less than the first stiffness.

6. The fluid-articulated structure of claim 5, wherein the reinforcing member of the inner sheath comprises a coil or braid with a first metal wire having a first diameter and the reinforcing member of the outer sheath comprises a coil or braid with a second metal wire having a second diameter smaller than the first diameter.

7. The fluid-articulated structure of claim 2, wherein the asymmetric plates are torsionally affixed to the inner sheath or the outer sheath or both by adhesive bonding, ultrasound welding, or thermal bonding.

8. The fluid-articulated structure of claim 1, wherein the channels of the asymmetric plates extend laterally to the aperture or a peripheral edge to facilitate insertion of the balloon strings therein.

9. The fluid-articulated structure of claim 1, wherein the balloon string comprises a first balloon string and wherein a second balloon string extends axially through channels of the asymmetric plates, each balloon string having a plurality of balloons extending between adjacent asymmetric plates and, when inflated, urging the first sides of the plates apart.

10. The fluid-articulated structure of claim 9, wherein the first and second balloon strings are in fluid communication so as to be inflated together at a common inflation pressure.

11. The fluid-articulated structure of claim 1, wherein the asymmetric plates are included in a proximal segment and have passages extending between the axial surfaces, the proximal segment configured to bend laterally in a first bending orientation oriented away from the first sides of the axial plates, and further comprising an inflation fluid supply tube extending axially through the passages and a distal segment having a balloon array in fluid communication with the inflation fluid supply tube so as to bend laterally in a second lateral bending orientation transverse to the first lateral bend orientation.

12. The fluid-articulated structure of claim 11, wherein the balloon array of the second segment is configured to bend laterally in a plurality of orientations, the inflation fluid supply tube having a plurality of lumens.

13. The fluid-articulated structure of claim 1, wherein each asymmetric plate has a plurality of passages and a plurality of inflation fluid supply tubes extend axially therethrough so as to allow bending at a plurality of axially offset segments.

14. A fluid-articulated structure comprising:
an outer sheath having a proximal end and a distal end with a lumen extending therebetween, the lumen of the outer sheath having a central axis;
an inner sheath disposed within the lumen of the outer sheath, the inner sheath having an inner lumen and an offset axis offset eccentrically relative to the central axis of the outer sheath;
an axial series of asymmetric plates disposed between the inner sheath and the outer sheath, the asymmetric plates having opposed axial surfaces, the axial surfaces having eccentric apertures receiving the inner sheath therethrough so the axial surfaces are primarily on a first lateral side of the plates relative to the inner sheath, the asymmetric plates having channels extending between the major surfaces on the first lateral side; and
a balloon string extending axially, the balloon string including an axial series of balloons separated by necks, the necks extending through the channels of the asymmetric plates and the balloons having axially oriented ends engaging the major surfaces on the first lateral sides of the asymmetric plates so that when the balloons are inflated the balloons urge the plates apart and bend the central axis of the outer sheath.

15. A method for bending a fluid-articulated structure, the method comprising:
inflating a balloon string including an axial series of balloons separated by necks, the necks extending through channels of a plurality of asymmetric plates, the asymmetric plates having eccentric apertures with an inner sheath extending therethrough, wherein the inflating is performed so as to urge axially oriented ends of the balloons against major surfaces of the asymmetric plates on first lateral sides of the asymmetric plates relative to the sheath, and wherein the balloons urge the first sides of the plates apart and bend a central axis of the inner sheath.
